(19) United States
(12) Patent Application Publication
(10) Pub. No.: US 2009/0143669 A1
(43) Pub. Date: Jun. 4, 2009

(54) COLOR MAPPED MAGNETIC RESONANCE IMAGING

(76) Inventors: Steven E. Harms, Fayetteville, AR (US); Scott Spangenberg, Amherst, NH (US); Xiaole Hong, Acton, MA (US)

Correspondence Address: DUANE MORRIS LLP - Philadelphia;IP DEPARTMENT, 30 SOUTH 17TH STREET, PHILADELPHIA, PA, 19103-4196, US

(21) Appl. No.: 12/328,543

(22) Filed: Dec. 4, 2008

Related U.S. Application Data
(60) Provisional application No. 60/992,308, filed on Dec. 4, 2007.

(51) Int. Cl. A61B 5/055

(52) U.S. Cl. 600/410

(57) ABSTRACT

A method and system estimate the risk of malignancy of a given region of interest using noninvasive MRI techniques. The determination of risk is based on the morphology and kinetic enhancement of a region of interest. In addition, the method and system use the type of the enhancement curve to determine the level of risk associated with a given region of interest. The region of interest can be a lesion, tumor, or other unknown. The imaging can be done with the aid of a contrast agent. Regions meeting component concentration criteria, time-change dynamic criteria and the like are distinctly colored and displayed on a single image. Different colors can be shown locally to identify predetermined levels of risk, and/or associated with predetermined compositions such as contrast agents, or to show the presence of silicone.

### FIELD OF THE INVENTION

The invention relates to techniques for processing and displaying the results of clinical imaging, particularly nuclear magnetic resonance imaging of the human breast for breast cancer screening, diagnosis and/or treatment.

### BACKGROUND

Lesion morphology and kinetic enhanced contrast analyses can provide information that may enable a skilled practitioner to assess the relative probability that a tissue anomaly or lesion might be malignant when seen in the nuclear magnetic resonance image (MRI) of a breast or other tissue. That is to say, an experienced practitioner or radiology technologist can learn to assess risk based on visual clues that are found in MRI images. There are various different ways that MRI images are obtained, weighted, enhanced by use of perfused agents or by image processing routines and otherwise produced. Thus, it is not clear how best to collect MRI data and to compare different collected images, to optimize the probability that dangerous lesions receive appropriate attention.

Among other techniques, a subject may be perfused with a contrast agent such as a gadolinium compound that binds more vigorously or over a longer time to certain tissue types versus other types. In a contrast study, a practitioner may obtain plural MRI images over time after perfusion and compare the extent to which the contrast produced by the agent affects some tissue structures more than others, or fades more quickly or more slowly from some tissue structures versus others.

Studies have found that a lesion displaying a washout kinetic contrast enhancement curve over time is more prone to be malignant than a lesion or anomaly that displays a distinctly different curve. This is one of the image clues available to a practitioner. However, a disciplined and effective method to identify a lesion using this type of clue has been elusive. It may be appropriate to adhere to a strict regimen of dosing and imaging conditions and timing to provide a basis for dependable data comparison. Handling the imaging results is cumbersome and generally requires a radiologist to register and compare selected points in several images to diagnose the lesions. An improved process of identifying lesions based on their morphology and kinetic enhancement curves would be desirable.

MRI relies on the relaxation properties of nuclei subjected to a steady state magnetic biasing field, excited by radio frequency signals, and caused to produce responsive electromagnetic radiation at locations that are addressed by timed gradient magnetic fields and phase relationships. The object is to collect data values that distinguish different types of tissue by location. A spatial resolution is needed to sample a minimum incremental volume of a size that is pertinent to tissue structure, and an amplitude resolution is needed to permit distinctions to be drawn between different types of tissue that are adjacent, due to differences in such amplitude.

Imaging data can be represented by mapping different data amplitudes to points in two or three dimensions. The different amplitudes can be represented by mapping a range of amplitudes to a range of luminance (brightness) levels over a gray scale. The mapped data is displayed in a graphical projection. For example, an image of tissues adjacent to a theoretical slice through the tissue can be shown in two dimensions.

In some applications, and especially for visualizing tissue structures within a three dimensional volume, it may be preferable to display some tissue types as relatively more opaque and to present other tissue types as being translucent or substantially transparent. This can reveal tissue structures, surface characteristic and the like for the elements presented as opaque. The data representing the tissue structures in the three dimensional imaged volume are projected by image processing routines onto a two dimensional display screen. Anatomical features are visualized by rotating the projection so as to view the projected volume from different perspectives and to bring rearward structures to the fore or out from a position where they are occluded by nearer structures.

The nuclei of atoms have magnetic moments. The magnetic moments can be aligned by application of a biasing magnetic field. Application of a radio frequency excitation signal at a resonant frequency for a particular element or isotope (known as the Larmor frequency) reorients the magnetic moments of the nuclei that correspond to that resonance frequency. The nuclei are tilted relative to their original alignment and tend to spin or precess as the nuclei come back into alignment with the biasing magnetic field over a period of time. By setting the excitation frequency to the resonance of a particular element nucleus, it is possible selectively to reorient and produce a spin echo signal specifically from the nuclei of element. Differences in tissue composition are made detectable.

For example, by exciting tissue at the hydrogen resonance frequency, tissues with high concentrations of water (H₂O) produce a more robust response than tissues that have a low water concentration. At a different resonance frequency, it is possible to excite a spin in nuclei other elements, such as elements that are concentrated in fatty tissues (lipids).

Various results are obtainable using different excitation pulse sequences to develop voxel values in three dimensions wherein the encoded value for each voxel represents a response of a particular element with respect to one or more parameters. The distinguishing parameters can be the resonant frequency of excitation, the amplitude of RF emission at the resonant frequency and/or differing from the resonant frequency, the rate of delay in dephasing and fading away of the echo response and other aspects that permit one element to be distinguished from another element and/or permit assessment of the relative concentrations of elements at different locations.

Distinct responses are useful not only for distinguishing the nuclei of different elements. In addition, the relative concentrations of two or more elements can be used to distinguish different tissue types. Using these responses, magnetic resonance imaging is useful to distinguish tissue types such as fat versus muscle or bone, and tissue structures such as blood vessels. Within a homogeneous tissue volume, MRI can distinguish areas of edema or ischemia, etc.

A given tissue type might be represented in a projected image by brightness, opacity, or color, or in video display terms, by luminance, saturation, and hue. Relative concentrations of elements also might be mapped to display brightness or other characteristics. For breast cancer diagnosis and some other uses, MRI projections are intended to assist in visualizing selected tissue structures rather than all tissue structures. Distinguishable tissue structures are accentuated and other tissue structures are darkened, faded or omitted in the projection. These are processed rather than realistic representations of the imaged volume

In the diagnosis and treatment of breast cancer, it is useful to distinguish tissue types and densities including concentrations of fat tissues versus concentrations of water. These distinctions enable visualization of internal breast tissue structures such as ducts and vasculature. Rendering fatty tissues transparent in a volume projection, or rendering the fatty tissue as dark in an image slice, tends to highlight ductal structures, to impart contrast to the image of lesions, to enable a practitioner to distinguish cysts from tumors, and so forth.

Contrast agents can be introduced to improve the extent to which pertinent tissue types and tissue structures can be distinguished, in particular because the contrast agents assume different concentrations in different tissues. Contrast agents with distinct nuclear magnetic characteristics (such as gadolinium) can be injected to enhance the contrast of particular tissue types, to limn the contours of blood vessels and other structures. Tissues also can be distinguished with respect to differences in the rates at which a perfused contrast agent diffuses into the tissues and the image contrast obtained by the contrast agent fades away in successive MRI images taken over a period of time.

In a NMR/MRI imaging arrangement, the subject is placed in a static magnetic field (a biasing field), and then excitation signals are applied to induce a response. Time varying magnetic fields (gradient fields) permit localized points in the tissue volume to be addressed. Sequences of radio frequency pulses excite nuclei that respond at specific RF pulse frequencies, typically reorienting the magnetic moments and spin axes of the nuclei from their initial orientation in the biasing field. Following a pulse that reorients the magnetic moments of adjacent nuclei, the nuclei relax over a period of time (T1) and return to their original alignment relative to the biasing field. The specific time period varies with the type of nuclei, the incident magnetic fields, and the amplitude of the excitation.

Adjacent nuclei of the same element, subjected locally to the same biasing field, gradient field and excitation conditions, have magnetic moments that tend to precess synchronously, in phase with one another, which persists for a limited time after the excitation is discontinued. The phase-synchronized spins of a group of adjacent nuclei reinforce, together producing a detectable spin echo signal at the resonant frequency. The amplitude of the signal varies with the concentration of nuclei that are precessing in phase. The signal can be resolved as to a corresponding location in a volume, i.e., a voxel value. The spin echo dies away over a period of time (T2) as more and more of the nuclei fall out of phase with one another and no longer reinforce. This time period is related to the type of nuclei, the bias conditions and excitation, and the temperature of the sample being imaged. When the phases are random, the net emitted spin echo signal is zero. Pulse sequences are designed to include pulses that are synchronous with nuclei of a particular element, and/or that affect the precession and phase relationships of adjacent nuclei. Signals are received along a phase encoding axis and are sampled, digitized and processed by Fourier transforms to convert so-called k-space data to spatially resolved amplitude data. The resulting values can distinguish the nuclei of one element from another in a three dimensional matrix of voxel locations. The values are stored in a memory referenced to spatial location in the imaged volume and can be displayed in slices or projections, enabling the practitioner to visualize the tissues, based on the detected concentrations of elements therein.

For contrast studies and other time-related techniques, plural successive images can be obtained, spaced by periods of time. These time-spaced additional images can be stored in the memory and the succession of values for a given voxel location, registered in memory from one image to the next, represents a fourth matrix dimension, namely time. Data processing software applied to the image data can be used to map data values to brightness and other video parameters, in selected slice planes or in projections of a volume that can be still frames or animated and are readily moved, magnified, rotated and otherwise manipulated in versions that are displayed.

In certain NMR/MRI arrangements, the gradient magnetic fields are placed and modulated to image thin slices of tissue that are perpendicular to a direction in which the patient is moved relative to the fields, in incremental steps between the imaging of successive slices. The collected data for the respective pixels in each slice are associated as a stack of slices. The spatial resolution of volume elements (voxel) corresponds to the X-Y resolution within a slice and the pitch spacing between successive slices. However, it is not necessary always to modulate the bias and gradient fields in an orthogonal X-Y and stepped Z raster-like progression of slices. In a different technique, the fields are modulated to target a succession of voxels in another pattern, such as a moving line or plane pattern that addresses all the voxel positions in a volume over a period of time. The data is collected in successive iterations, and the voxel resolution is related to the imaging time. It is desirable to collect an image quickly, but it is also desirable to provide a fine resolution, which interests involve a trade off. It is generally necessary to reach a compromise of image collection time versus resolution.

A spiral imaging technique is exemplified by commonly owned U.S. Pat. Nos. 5,202,631; 5,304,931; and 5,415,163. The disclosures of these patents are incorporated by reference herein in their entireties. In a spiral imaging arrangement, a spiral trajectory is defined in an X-Y plane for a phase encoding line extending in the orthogonal Z axis. After completing a spiral trajectory or shot, the spiral is moved, e.g., incrementally rotated, and in the next shot follows a trajectory that fills in k-space data positions that are different from those of the initial shot, for example filling in positions between the spiral bands of the next previous shot. There is a tradeoff in balancing the needs for fine spatial resolution to distinguish small anatomical structures, versus a short image collection time to demonstrate the progress of an event such as wash-in and out of a contrast agent over a period of time.

### SUMMARY OF THE INVENTION

It is an object of the invention to analyze and employ MRI intensity data using image data processing and display generation routines to derive a measure of likely risk from the information found in one or plural time-spaced MRI images, preferably plural images demonstrating the effects of a perfused contrast agent over time. The intensity data is analyzed for shapes and/or contrast agent fading characteristics to generate new parameter values associated with risk. The results are at least color coded to accentuate structures of regions of particular risk. Preferably, the risk is assessed over a range and the risk level is mapped, voxel by voxel by a display generator, to a range of display color and brightness characteristics that correspond to the range of risk. The color encoded images are displayed as an overlay to planar slices or 3-D volumes. The risk levels can be mapped to colors over a scale of cool to hot colors for increasing levels of risk.

Accordingly, a method and system have been developed to display the estimated risk of malignancy of a given region of interest using noninvasive MRI techniques. The determination of risk is based on the morphology and kinetic enhancement of a region of interest. In addition, the method and system use the type of the enhancement curve to determine the level of risk associated with a given region of interest. The region of interest can be a lesion, tumor, silicone implants, tissue anomaly or other unknown or indefinite region to be examined. The imaging is done with the aid of known contrast agents.

Using image processing techniques to distinguish aspects of shape and using a comparison of the levels of contrast at a given registered voxel position or region over time, expert rules are applied to assess the level of risk according to a scale of probability of malignancy and/or the gravity of the perceived risk. Once the level of risk is determined for a given region, the region can be marked and displayed on a single image that summarizes the level of risk that preferably was assessed using a time sequence of several images.

According to another aspect, the voxels in the image are displayed not only using contrast, but in addition, the risk level associated with spatial points and tissue structures appearing in the image can be mapped to a range of colors that demonstrate the perceived level of risk. Generally, alarm or hot colors include red, magenta, orange, yellow, etc., and are associated with higher levels of risk. Grayscale can be associated with regions of moderate or unknown risk (e.g., regions of weak enhancement). Cool colors such as blue, turquoise, and green are associated with a lower risk (no enhancement or negative enhancement). Display of voxels in this context includes all manner of presentation such as presentation as a planar slice of pixels, projection of a volume where three dimensional voxels are shown from a point of view in an image having two dimensions, rotation, magnification and other alterations of presentation, etc. The display also can comprise supplemental or superimposed graphics such as topographical graphs. The displayed features can be associated with indicators, blinking points or regions. The display of colors and other risk aspects can be selectively switched on and off, or the display can be selectively switched to a different view of the corresponding area, such as different weighted images that show alternative depictions of the same areas.

The invention is advantageous for meaningful display of MRI data with respect to risk, and in particular risk of malignancy for identified points, tissue structures or regions. The system and method also may be used in other ways, for example for parametric color mapping to identify silicone in implants or in leakage from implants, which can be displayed in a distinct predetermined color. To recognize silicone, a silicone-suppressed image can be subtracted from a non-silicone suppressed image and the resultant image can be color coded for silicone in regions that show a detectable presence or a given concentration of silicone. Risk of malignancy can also be assessed in part using precontrast images, especially to improve the contrast associated with some features by subtracting away from the post contrast data at least part of the precontrast image data so as to remove the common mode background image from the displayed characteristics. It is also possible to use a reference image wherein the spatial resolution or T1 and T2 characteristics enable discrimination for compositions such as silicone, in which case subtraction may not be needed. Also, information obtained from subtraction or from a reference image can be supplemented by curve shape information.

### BRIEF DESCRIPTION OF THE DRAWINGS

The drawings show certain embodiments of the invention that are discussed herein as examples. However the invention is not limited to these examples. Reference should be made to the appended claims to determine the scope of the invention. In the drawings,

FIG. 1 is a plot the shows aspects of a steady enhancement curve with time plotted along the horizontal axis and intensity plotted along the vertical axis. The intensity value can be the MRI resolved intensity value associated with a voxel, an average over a region of voxels, or a distinctly identified tissue structure or part thereof.

FIG. 2 likewise shows a plateau enhancement curve with time plotted along the horizontal axis and intensity plotted along the vertical axis.

FIG. 3 shows a washout enhancement curve with time plotted along the horizontal axis and intensity plotted along the vertical axis.

FIG. 4 shows a block diagram representing the steps taken according to an exemplary embodiment of the method. More or fewer steps may performed in practicing other embodiments.

FIG. 5 shows a resulting MR image that is shaded to represent color mapping. In this case, the shaded area represents red, a high risk lesion. The un-shaded areas represent parts of the image that have not been color mapped or have been rendered as low or neutral risk.

### DETAILED DESCRIPTION

According to an inventive aspect, imaging data characteristics obtained by nuclear magnetic resonance imaging (MRI) techniques, especially the time-changing effects of perfused contrast agents on the intensity of voxel points in imaged breast tissues. The data are processed to evaluate the risk associated with a given object within the breast according to one or more diagnostic standards that are preferably automated and accomplished in image data processing routines. The processed level of risk perceived for respective voxel points, tissue structures and/or imaged regions, is mapped to the display of intensity as used for visualizing the tissue structures.

In one embodiment, the amplitude of the MRI response is mapped to the intensity of the image of the corresponding tissues structures. The associated level of risk for the tissue structures or distinct areas thereof, is mapped to a distinct image attribute associated with the displayed image of that tissue structure or area. Thus, when a risk assessment produces a display in which a particular tissue structure is shown with a concentration of risk-representing voxel points, such as a lesion, the display draws appropriate attention to the lesion as a risk.

In an apt example for breast cancer diagnosis, two factors can be used to determine the risk associated with an object in the breast, namely morphology and kinetic enhancement curves. Morphology characteristics (e.g., the shape of identifiable tissue structures) can be detected from precontrast data but preferably such characteristics are enhanced by perfusion of the subject with a contrast agent such as a gadolinium compound that has a propensity to bind to different tissues in different concentrations, or for a longer or shorter period of time. The change in such concentration over time is measured and regarded as a kinetic or time-varying contrast enhancement characteristic for each respective voxel position or local grouping of voxels in successive MRI images collected at successive times, as registered with one another to align the positions of voxels from one image to the next.

Morphology concerns the shape of a lesion or anomaly in the MRI image data. Exemplary aspects of morphology that are pertinent include irregular masses with spiculated borders, masses with peripheral enhancement, and masses with ductal enhancement, which aspects are associated with increased risk of malignancy. Lesions and anomalies that generally are considered a low risk for malignancy are likely to be characterized by smooth lobed borders, low contrast enhancement, and/or patchy parenchymal enhancement.

Kinetic enhancement curves also are believed to reflect the risk posed by a given lesion. Kinetic enhancement curves represent the rate the contrast agent flows through a given tissue and can be correlated to a specific voxel or location of the MRI. From the kinetic enhancement curves, the type of tissue imaged can be determined. The type of tissue present can be strongly indicative of the risk for malignancy. Consequently, the risk associated with a given lesion can be estimated. The intensity data used to create the enhancement curves can also be displayed on each sequential MRI slice using colors or the intensity of a black and white image.

Kinetic enhancement curves are generated from the enhancement (or suppression, i.e., negative enhancement) of the MR signal at a given voxel over time after the contrast agent is injected into the patient's breast. Kinetic enhancement curves can be based on T1 or T2 methods. The T1 method shows a contrast medium outside of the cells and may demonstrate micro-vessel profusion and extracellular leakage space. The T2 method shows contrast medium in the vascular phase and indicates tissue perfusion and blood concentration in a given area.

Kinetic enhancement curves generally have one of three shapes: (1) steady enhancement, (2) plateau, and (3) washout. Each curve shape represents a different type of tissue with a relative associated risk of malignancy. The enhancement curves are plotted, with time on the x-axis and local or regional voxel intensity on the y-axis. The regional voxel intensity can be measured within the boundaries of a morphological tissue structure identified using edge detection techniques. The overall intensity of the enhancement generated in the region by the addition of the contrast agent also is indicative of the risk that the region poses. Stronger enhancement values for both a feature and a region are more indicative of a malignant lesion or region than a lesser enhancement value.

Steady enhancement curves (persistent enhancement curves) generally increase overtime, meaning the MR signal intensity increase as time increase after the injection of the contrast agent. Studies show that steady enhancement curves are indicative of benign lesions or of lower risk lesions.

Plateau enhancement curves as the name suggests are shaped like a plateau. The plateau curves quickly reach a maximum intensity which levels off, maintaining a relatively constant intensity through time. Lesions with plateau type enhancement curves tend to be a moderate risk of malignancy.

Washout enhancement curves quickly reach a maximum intensity after the contrast agent is injected. The intensity decrease over time as the contrast agent fades. Washout curves are considered to be representative of the most malignant lesions.

Both qualitative and quantitative methods of analysis may be used to determine the type of enhancement curve exhibited. The intensity data can also be displayed as dynamic data on each successive 2-D slice of the generated MRI through time. The intensity data could be displayed using the brightness value of the black and white image or the color associated with an intensity value. In the past, radiologist would have to compare multiple images to classify the lesion. The multiple images would include color intensity images and black and white images, which would increase the effort and time required for the radiologist to correctly classify the tumor. In addition, it was an ungainly process that was susceptible to human error and confusion. Consequently, an improved method of representing the available data was needed to accurately and efficiently classify lesions based on morphology and kinetic enhancement curve data.

Radiologists thus are provided with a form of information respecting lesion structure and the shape of the enhancement curve for a given region of interest to interpret the health of breast tissue. The invention also uses parametric color mapping to classify tissues by distinguishing among their responses to phase spoiling. An exemplary embodiment of the invention uses parametric color mapping to classify and segment tissue according to the shape of the curve that describes the dynamic response of breast tissue to the injection of a contrast agent over the course of a breast MRI study that may involve a number of time spaced images, images obtained with different resonance frequencies and MRI weighting arrangements, etc.

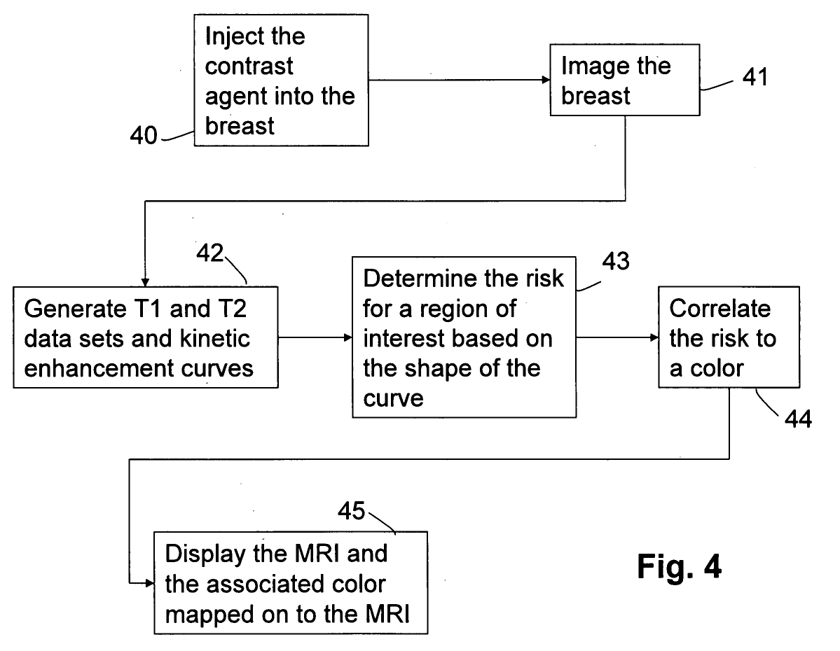

An embodiment comprising a method for displaying the estimated risk of malignancy of a given region of interest, using noninvasive magnetic resonance imaging relying on morphology and kinetic enhancement of the region of interest, is generally shown in the block diagram of FIG. 4. This embodiment comprises injecting a subject with a contrast agent 40 and imaging the breast 41 over a period of time during which time-spaced images of the same tissue volume are collected and the time change in MRI intensity due to the local concentration of the contrast agent is monitored to produce a time contour. A precontrast image can be included as a reference or to provide a background common mode image that is subtracted to enhance the distinct levels of intensity of the contrast agent in different tissues.

The contrast agent can be injected locally or as a bolus that circulates in the vascular system. Various contrast agents are known that allow T1 and T2 datasets to be generated, from which MRI intensity data is obtained. The contrast agent kinetics vary among different types of tissue in which the contrast agent is perfused, and vary with time in that the contrast agent diffuses over time and local variations in contrast diminish more or less and more quickly or more slowly.

The contrast agent can be employed positively or negatively to enhance the appearance of a region displayed on an MRI or to suppress it. A contrast agent will enhance or negatively enhance a region displayed on an MRI, and the region will be enhanced at varying intensities through time. Since different tissues are affected differently over time, an image processing routine, like an experienced radiologist, can categorize different tissues based on their reaction to a contrast agent and the enhancement over time.

Once the breast has been imaged (block 41) and the necessary data is collected, or while the breast is being imaged in an ongoing repetitive sequence, one or both of a T1 dataset a T2 dataset are created. An enhancement curve is generated at block 42, based on the intensity over time of the MRI response obtained at distinct imaged locations (voxels). In the case of multiple images, a voxel position in a later image is registered to a voxel position in an earlier image, either by assuming an unchanged location in the imaged voxel matrix or by conventional image stabilization techniques. The MRI intensity data for a given voxel position, or for a region encompassing plural voxels, changes over time for the breast tissue that was subjected to the contrast agent. This change in intensity is monitored to determine its contour over time and compared to stored values to distinguish certain signature time change contours.

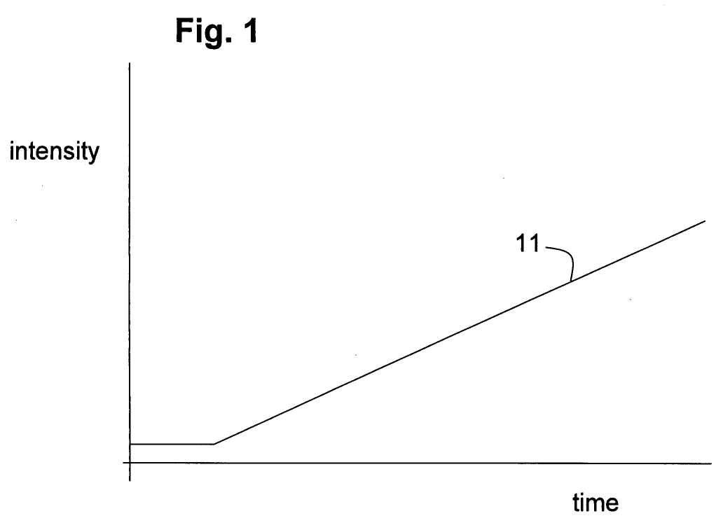

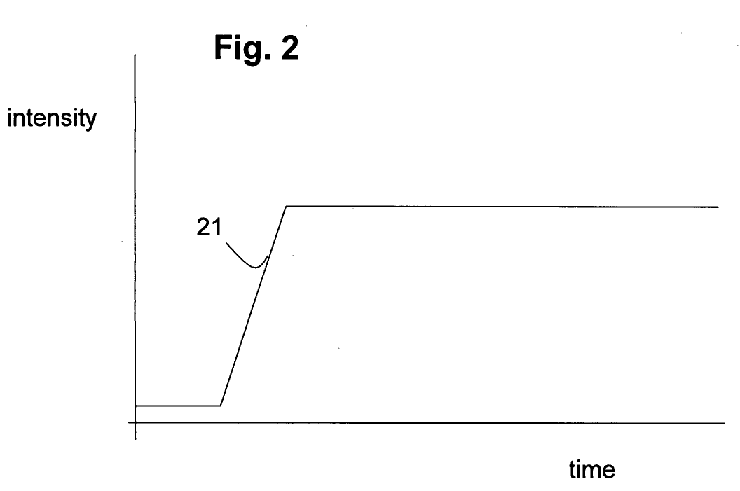

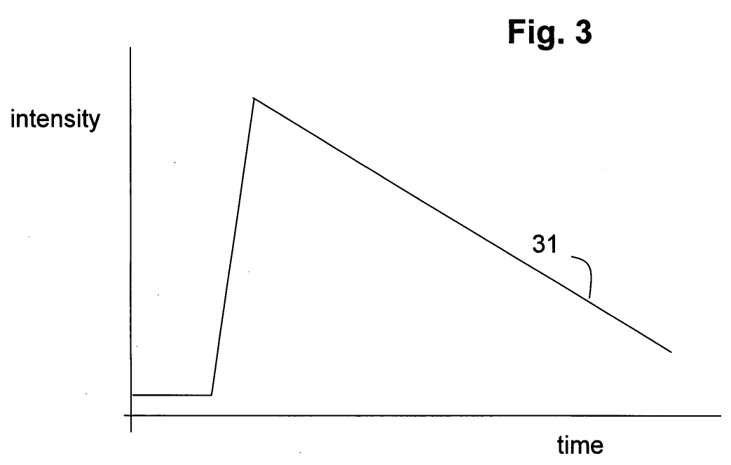

The shape of the time-change curve typically falls into one of three forms, examples of which are shown in FIGS. 1-3. The shape of the curve is associated with a level of risk of malignancy for tissues comprising the voxel or associated with the region for which the curve was generated. FIG. 1 shows a steady enhancement curve 11 that is indicative of a moderate to low risk of malignancy. FIG. 2 shows a plateau enhancement curve 21 that is indicative of a moderate risk of malignancy. The contour shown in FIG. 3, however, is a so-called washout curve 31, and is generally associated with a relatively higher risk of malignancy for a given region.

It is possible by image processing techniques to obtain contour information for every voxel in the imaged tissue or in a region of the imaged tissue. This region can be selected by image processing techniques, for example by identifying an area of contrast within defined perimeters, and regarding the area within the perimeters as a distinct structure to be treated as a region. Alternatively, the practitioner can select a tissue structure to be analyzed in this way.

The region that is of interest is typically a lesion. The morphology of the lesion or region including its shape and the character of its edges can be recorded and also play a factor in contributing to an overall assessment of the risk of malignancy. Assessing the morphology of a lesion is an expert skill and can be accomplished with the attention of an attending radiologist. Alternatively, an image analysis process can be employed to scan an image for pertinent feature such as areas of contrast with indistinct edges, and either automatically to select one or more associated voxel or regions for analysis or to indicate the location of areas to be brought to the attention of the radiologist. It is possible to incorporate criteria related to morphology into the risk assessment. However for simplicity it will be assumed from this point forward that the morphology is used (manually or automatically) to select a region to be analyzed for the kinetic time change in enhanced intensity, and that the color mapping of the selected region is based only on the contour of the time change.

A time-change intensity contour can be obtained for an average area, for selected voxel points, for an array of voxel points, for all voxels within a certain distance of a reference, etc. A curve can be created and plotted if desired (block 43), or the successive data values can be applied to a mathematical regression routine to assess whether and optionally how strongly the time-changing intensity matches one of the curves that are regarded as associated with a low, medium or high probability of malignancy.

The classification of the shape of the curve can involve a quantitative or qualitative assessment. A quantitative assessment can be accomplished by a direct comparison of intensities in corresponding registered voxels from successive images. Alternatively, the intensities can be preprocessed by normalization or automatic gain control. The total enhancement or negative enhancement produced by the contrast agent in a given tissue region (i.e., the average level) can be included in the determination of risk, because a greater enhancement caused by the contrast agent through time can be an indication of a higher risk.

The time-change contour determination can be done for a series of voxels, a single voxel, an identified tissue structure, a region of the image, a region of a tissue structure, etc., namely for any pertinent region that has been imaged by one or more magnetic resonance imaging techniques. Given a curve shape over time that is associated with a known risk, for example as in FIGS. 1-3, the risk level is identified by displaying the corresponding voxel, tissue structure or region in a color 44 that is specific to the level of risk. The respective colors can be made selectable to indicate a level of risk using user-preferred colors. Preferably, however, the association of color with risk level is standardized to comprise typically-used warning colors to represent the extent of risk. For example, the color temperatures for low, medium and high risk can be assigned such that increasing color temperature is associated with increasing risk to the patient. That is, a range of hot colors (such as yellow, magenta, red) can be assigned where there is strong enhancement of tissue. Grayscale colors can be assigned to areas where there may be some enhancement of tissue but not strong enhancement. Grayscale colors alternatively or additionally can be applied to tissue types for which the user may have disabled a specific color mapping, thereby reducing the complexity of the display and/or increasing the prominence of colors that remain. Cool colors (such as blue, turquoise, green) can be assigned to areas where there is negative enhancement, whether strong or weak. In addition to distinctions concerning the extent of enhancement (e.g., strong enhancement, minimal or no enhancement, negative enhancement), distinctions may be drawn based upon dynamic aspects such as curve shape (persistent enhancement 11, plateau enhancement 21, and washout enhancement 31).

The radiologist depends on information about tissue structure and dynamic behavior of lesion tissues. Therefore, the specific colors applied in the color mapping are chosen from the palette of possible colors for their ability to preserve the display of tissue structure transparently. This enables the risk-encoded image to be used for visualizing tissue structures and is assisted by the radiologist experience in classification of lesions according to their structure and morphology, and does not require the radiologist to turn the color mapping on to show enhancement level and then off to better reveal tissue structure. Therefore, for example, white as an alarm color may be less desirable than red, magenta or yellow, which are more effective than white to demonstrate tissue structure (e.g., by variations in shading).

Enhancement curves are shown in FIGS. 1-3 for illustration purposes. The measured enhancement curves need not be displayed to the user if the resulting color-risk encoded images are displayed in a manner that presents distinctions in dynamic behavior at least partly by differences in color coding. However, graphic plots of the curves can be available when requested so the radiologist can ensure the risk factors were accurately determined, and perhaps also discover some other useful factor in the plot or in the raw data on which the risk determinations were based. Because the criteria for distinction between curve shapes can sometimes be ambiguous (e.g., washout versus plateau, or plateau versus persistent), it is advantageous to present color mapping on the plot of the curve as well as on the image. This reveals how the color mapping seen in the image relates to the shape of the plot.

After the color mapping has been applied to a region based on the risk that the region is deemed to present, the image is displayed (block 45) showing higher risk areas in higher risk colors. The image can be displayed on a single image and eliminates the need for a radiologist to compare two images. The single color-encoded displayed image obviates the need to compare relative brightness that would normally be necessary to compare levels of enhancement in successive black and white images. The technique also eliminates the need to identify particular regions of interest by added graphics such as boxes or arrows or blinking, etc., because the color mapping is generally sufficient to identify the highest risk curve shape associated with a lesion.

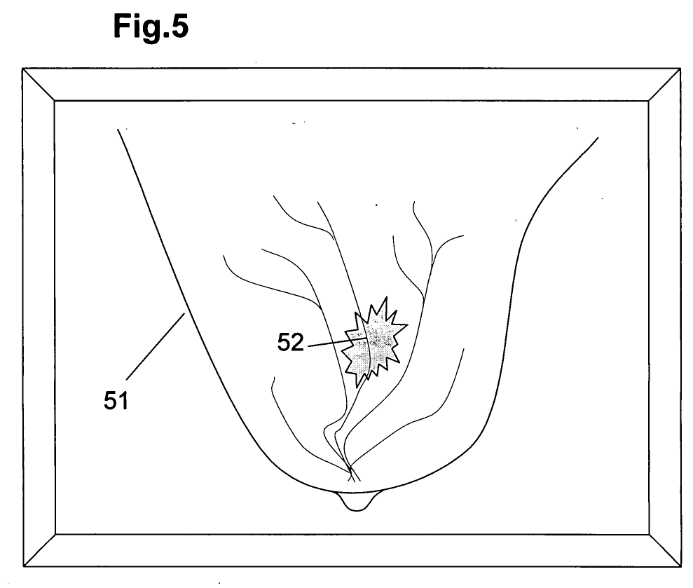

FIG. 5 shows a representative display, namely the projected volume of an imaged breast 51. The region of interest 52 is shaded. The shading represents the color red, to indicate a high risk, based on a high intensity washout-curve for the associated lesion.

In one embodiment the radiologist selectively controls when color mapping is applied or not applied, the particular images or areas to which color mapping is applied, and which enhancement curve patterns are color-mapped. Additionally, the palette of color choices that are offered can be colors and shades that are able to display tissue structure transparently. In this way the color mapping remains a useful tool but does not overwhelm the radiologist. The color mapping also helps to retain the structural information about the lesion or region that is displayed in the MRI presentation. When the color mapping is selectively removed from the image, the radiologist can use the intensity data in conventional ways.

Color mapping that does not preserve the ability to see tissue structure means that a radiologist must display acquisitions with and without color mapping in order to classify lesions. This increases the effort required by the radiologist to interpret images, and it makes less efficient use of the available screen space. For grayscale display of images, the same brightness value may be applied to the Red, Green, and Blue channels. On the other hand, an embodiment of the present color mapping design involves applying the same brightness value used for grayscale display to single color channels (red, green, and blue) and pairs of color channels (red and green for turquoise, red and blue for magenta, and green and blue for yellow). This allows the structure of tissue and variations in MRI response intensity to be seen through the color mapping. Mapping to additional 24 bit colors is not necessary and may obscure tissue structure or introduce the appearance of structure that does not correlate with the structure apparent in grayscale mapping.

The color mapping and risk determinations can be done with the use of a computer system with software that when executed performs the steps of the present invention or can be done through other processing means suitable for performing the invention. In addition, the MRI can be performed by any MRI system that will report the necessary intensity and image data in a form that a processing system can manipulate. The display of the data can be on a color screen compatible with the other components. The displayed data can also be stored and saved for later use with or without the color mapping.

In another embodiment, post-contrast-only RF spoiling and negative enhancement are used to image an area suspected to contain silicone. If the area actually contains silicone, the imaging system can display the silicone in voxels that are identified by a representative color.

Application of RF spoiling to spiral off-resonance (RODEO) images results in images that are both T1- and T2-weighted. By applying RF spoiling only to post-contrast RODEO acquisitions, one is able to discriminate cysts, which will be bright pre-contrast and dark post contrast from malignant lesions with a similar structure, which will be brighter post-contrast than they are pre-contrast, without having to include an additional T2-weighted acquisition and therefore lengthen the study. This also produces curve shapes (involving negative enhancement) that can be used to provide color mapping for free water (such as would be found inside a cyst) and protein-bound water (such as would be found in edema or healthy glandular tissue.) By combining voxel information from a pre-contrast, T1-weighted acquisition with the fat suppression that is intrinsic to RODEO imaging with negative enhancement and curve shape, color mapping of silicone within the breast is achieved.

Some preferred embodiments include the additional steps of imaging the breast before injecting it with a contrast agent, enabling image processing to compare pre and post contrast images and optionally to subtract a baseline image from an image taken with contrast and thereby to enhance the appearance of the contrast agent. The disclosed techniques, although useful to distinguish tissue types based on water concentrations, are also useful to respond to the concentration of compositions such as contrast agents that help to limn and distinguish tissue structures and also other pertinent compositions such as concentrations of silicone, which are not tissue or associated with tissue structures.

The invention has been described with respect to a number of aspects and embodiments that are believed representative. The invention is not limited to the particular embodiments and arrangements disclosed as examples. Reference should be made to the appended claims and not the foregoing discussion of examples, to assess the scope of exclusive rights claimed.

What is claimed is:

1. A method for displaying a risk assessment for a region of interest in a patient's tissues, comprising:
imaging a part of a subject's body using magnetic resonance imaging, to produce at least one volume image containing intensity data for a matrix of voxels;
identifying at least one of the voxels in an area of interest;
applying a test to the intensity data and determining a risk level from the test, for the at least one voxel from a plurality of possible risk levels;
correlating said risk level to one of a plurality of colors associated with the possible risk levels, wherein the plurality of colors represent levels of risk; and
displaying said volume image in a format wherein a displayed intensity of at least one of said at least one of the voxels and the area of interest is shown in said one of the plurality of colors, thereby displaying the intensity data and the risk level from the test.

2. The method of claim 1, comprising imaging the part of the subject's body repeatedly over time to provide multiple data sets, each said data set comprising at least one said volume image, wherein the at least one voxel is referenced in at least two of the data sets, wherein the test comprises matching a change in the intensity data between the at least two of the data sets.

3. The method of claim 2, wherein the risk assessment comprises estimating a risk that a breast tissue structure is malignant, further comprising applying a contrast agent prior to obtaining the at least two of the data sets, and wherein the test comprises matching the change in the intensity data to identify one of steady enhancement over time, a plateau in time and an enhancement washout.

4. The method of claim 3, wherein said imaging comprises generating a T1 data set and a T2 data set at least at three points in time, and applying the test comprises comparing the change in intensity data to criteria including at least three kinetic enhancement curves.

5. The method of claim 1, wherein the test distinguishes the risk level by at least one factor selected from the group consisting of qualitative kinetic enhancement curves, quantitative kinetic enhancement curves, and morphology of the region of interest.

6. The method of claim 5, further comprising determining the region of interest to correspond to a tissue structure and wherein the at least one voxel is one of a single voxel in the region of interest, a plurality of selected voxels in the region of interest and substantially all the voxels in the region of interest.

7. The method of claim 1, wherein the risk level corresponds to a level of probability that an imaged part of the patient's tissues is malignant, and the color representing the possible risk levels range from subjectively cool to hot colors for representing increasing levels of risk of malignancy.

8. A programmed system for determining and displaying an estimation of a risk of malignancy of a given region of interest in a magnetic resonance imaged volume of a subject, the system comprising:
an imaging system for producing a magnetic resonance imaging data set, coupled to a computer system with a memory for storing MRI data sets representing volume images, wherein the computer system is coupled to a display and is operable to generate a visual presentation of intensity values for one of a planar slice and a planar projection of a three dimensional matrix for visualizing tissue structures in the imaged volume;
a computer program code embodied in tangible computer readable storage media, that when the computer program code is loaded into and executed by a general-purpose processor, the computer program code directs the processor for:
imaging a part of a subject's body using magnetic resonance imaging, to produce at least one volume image containing intensity data for a matrix of voxels;
identifying at least one of the voxels in an area of interest;
applying a test to the intensity data and determining a level of risk of malignancy from the test, for the at least one voxel from a plurality of possible risk levels;
correlating said risk level to one of a plurality of colors associated with the possible risk levels, wherein the plurality of colors represent levels of risk; and
displaying said volume image in a format wherein a displayed intensity of at least one of said at least one of the voxels and the area of interest is shown in said one of the plurality of colors, thereby displaying the intensity data and the risk level from the test.

9. The programmed system of claim 8, comprising imaging the part of the subject's body repeatedly over time to provide multiple data sets to be stored in the memory, each said data set comprising at least one said volume image, wherein the at least one voxel is referenced in at least two of the data sets, wherein the test comprises matching a change in the intensity data between the at least two of the data sets.

10. The programmed system of claim 9, wherein the multiple data sets contain successive images obtained after perfusion with a contrast agent, and wherein the test comprises matching the change in the intensity data to identify one of steady enhancement over time, a plateau in time and an enhancement washout.

11. The programmed system of claim 10, wherein said imaging comprises generating a T1 data set and a T2 data set at least at three points in time, and applying the test comprises comparing the change in intensity data to criteria including at least three kinetic enhancement curves.

12. The programmed system of claim 10, wherein the test distinguishes the risk level by at least one factor selected from the group consisting of qualitative kinetic enhancement curves, quantitative kinetic enhancement curves, and morphology of the region of interest.

13. A method for displaying the estimated risk of malignancy of a given region of interest using a magnetic resonance imaging technique based on a morphology and kinetic enhancement time change of intensity of the region of interest comprising:
injecting the subject's body with a contrast agent;
imaging and re-imaging a part of a subject's body to obtain image data representing changing intensity over time;
comparing kinetic enhancement time change for at least one voxel in the image data, against a stored set of reference kinetic enhancement curves, wherein at least two of the reference kinetic enhancement curves have characteristics typical of different risks of malignancy;
determining from said comparing a risk level for a tissue area corresponding to the at least one voxel;
correlating the risk level for the tissue area to a selected color among a range of colors that identify distinct levels of risk; and,
displaying the at least one image derived from the image data wherein an intensity at the tissue area is represented by displayed image intensity and said risk level at said tissue area is represented by the selected color.
